(19) United States
(12) Patent Application Publication
(10) Pub. No.: US 2002/0107236 A1
(43) Pub. Date: Aug. 8, 2002

(54) Methods of treating sexual dysfunction associated with hypertension

(76) Inventor: Pritam Singh Sahota, New Providence, NJ (US)

Correspondence Address: THOMAS HOXIE, NOVARTIS CORPORATION, PATENT AND TRADEMARK DEPT, 564 MORRIS AVENUE, SUMMIT, NJ, 079011027

(21) Appl. No.: 10/008,445

(22) Filed: Dec. 3, 2001

Related U.S. Application Data
(60) Provisional application No. 60/250,540, filed on Dec. 1, 2000.

(51) Int. Cl. A61K 31/554; A61K 31/549; A61K 31/44; A61K 31/21; A61K 31/137

(52) U.S. Cl. 514/211.07; 514/223.2; 514/355; 514/509; 514/649

(57) ABSTRACT

The present invention relates to methods of treating SD associated with hypertension and another condition by administering a pharmaceutical combination of an angiotensin receptor blocker with either an anti-hypertensive drug or an HMG-CoA reductase inhibitor.

### BACKGROUND

Sexual dysfunction (SD) is more commonly observed in hypertensive patients especially those with diabetes and/or hyperlipidemia. Further, many commonly used anti-hypertensive drugs such as diuretics and beta-blockers can interfere with sexual function in both sexes, causing loss of libido, impairment of erectile function and ejaculation in men and delay or prevent orgasm in women. A specific angiotensin receptor blocker or antagonist (ARB), losartan, has been show to have an advantage in preservation of sexual function when used clinically for the treatment of hypertensive disorder in male rats. Chan et al., Pharmacology, Vol. 58, No. 3, pp. 132-139 (1999). It has also been suggested that administration of ARBs result in smooth muscle relaxation and thus erection in an anesthetized dog. Kifor et al., J. Urol., Vol. 157, No. 5, pp. 1920-1925 (1997). However, heretofore, there has not been a suitable treatment for SD associated with hypertension. Because of low response (40-55% efficacy) to anti-hypertensive monotherapy, combination therapy for hypertension (>80% efficacy) has to be used in a large number of patients.

Accordingly, there is a need for a method of treating a patient suffering from SD associated with hypertension comprising administering a therapeutically effective amount of a pharmaceutical combination to the patient, wherein the pharmaceutical combination comprise as active ingredients:

(i) an ARB or a pharmaceutically acceptable salt thereof; and

(ii) (a) an anti-hypertensive drug or a pharmaceutically acceptable salt thereof or (b) a statin or a pharmaceutically acceptable salt thereof. The pharmaceutical combination may be administered as a pharmaceutical composition comprising the pharmaceutical combination and a pharmaceutically acceptable carrier.

There is also a need for a method of treating a patient suffering from SD associated with hypertension and another condition, including but not limited to diabetes and hyperlipidemia comprising administering a pharmaceutical combination to the patient, wherein the pharmaceutical combination comprise as active ingredients:

(i) an ARB or a pharmaceutically acceptable salt thereof; and

(ii) (a) an anti-hypertensive drug or a pharmaceutically acceptable salt thereof or (b) a statin or a pharmaceutically acceptable salt thereof.

### SUMMARY

Toward these ends, and others, an aspect of the present invention provides a method of achieving a therapeutic effect for treating a patient suffering from SD associated with hypertension comprising administering a therapeutically effective amount of a pharmaceutical combination comprising as active ingredients:

(i) an ARB or a pharmaceutically acceptable salt thereof;

(ii) (a) an anti-hypertensive drug or a pharmaceutically acceptable salt thereof or (b) a statin or a pharmaceutically acceptable salt thereof to a patient in need thereof.

In another embodiment of the present invention there is provided a method of achieving a therapeutic effect for treating a patient suffering from SD associated with hypertension and another condition, including but not limited to diabetes and hyperlipidemia comprising administering a pharmaceutical combination to the patient, wherein the pharmaceutical combination comprise as active ingredients:

(i) an ARB or a pharmaceutically acceptable salt thereof; and

(ii) (a) an anti-hypertensive drug or a pharmaceutically acceptable salt thereof or (b) a statin or a pharmaceutically acceptable salt thereof to a patient in need thereof.

Other objects, features, advantages and aspects of the present invention will become apparent to those of skill from the following description. It should be understood, however, that the following description and the specific examples, while indicating preferred embodiments of the invention, are given by way of illustration only. Various changes and modifications within the spirit and scope of the disclosed invention will become readily apparent to those skilled in the art from reading the following description and from reading the other parts of the present disclosure.

### DESCRIPTION

The term “synergistic” as used herein means that the effect achieved with the methods and compositions of the present invention is greater than the sum of the effects that result from methods and compositions comprising the active ingredients of this invention separately.

The term “statin”, where used in the specification and the appendant claims, is synonymous with the terms “3-hydroxy-3-methylglutaryl-Coenzyme A reductase inhibitor” and “HMG-CoA reductase inhibitor.” These three terms are used interchangeably throughout the specification and appendant claims. As the synonyms suggest, statins are inhibitors of 3-hydroxy-3-methylglutaryl Coenzyme A reductase and, as such, are effective in lowering the level of blood plasma cholesterol. Statins and pharmaceutically acceptable salts thereof are particularly useful in lowering low-density lipoprotein cholesterol (LDL-C) levels in mammals, and particularly in humans.

In accordance with an aspect of the present invention there is provided a method of achieving a therapeutic effect for treating a patient suffering from SD associated with hypertension comprising administering a therapeutically effective amount of a pharmaceutical combination comprising as active ingredients:

(i) an ARB or a pharmaceutically acceptable salt thereof;

(ii) (a) an anti-hypertensive drug or a pharmaceutically acceptable salt thereof or (b) a statin or a pharmaceutically acceptable salt thereof to the patient.

In another embodiment of this aspect of the present invention the therapeutic effect achieved is synergistic, in that, the therapeutic effect is greater than the sum of the therapeutic effect achieved by the administration of the active ingredients separately.

In another embodiment of the present invention there is provided a method of achieving a therapeutic effect for treating a patient suffering from SD associated with hypertension and another condition, including but not limited to diabetes and hyperlipidemia comprising administering a pharmaceutical combination to the patient, wherein the pharmaceutical combination comprise as active ingredients:

(i) an ARB or a pharmaceutically acceptable salt thereof and

(ii) (a) an anti-hypertensive drug or a pharmaceutically acceptable salt thereof or (b) a statin or a pharmaceutically acceptable salt thereof to the patient.

In another embodiment of this aspect of the present invention the therapeutic effect achieved is synergistic, in that, the therapeutic effect is greater than the sum of the therapeutic effect achieved by the administration of the active ingredients separately.

ARBs (which are called AT₁-receptor antagonists and angiotensin II receptor antagonists) are understood to be those active ingredients which bind to the AT₁-receptor subtype of angiotensin II receptor but do not result in activation of the receptor. As a consequence of the inhibition of the AT₁-receptor, these antagonists can, for example, be employed as anti-hypertensives or for treating congestive heart failure.

The class of ARBs comprises compounds having differing structural features, essentially preferred are the non-peptidic ones. For example, mention may be made of compounds selected from the group consisting of valsartan, losartan, candesartan, eprosartan, irbesartan, saprisartan, tasosartan, telmisartan, the compound with the designation E-1477 of the following formula

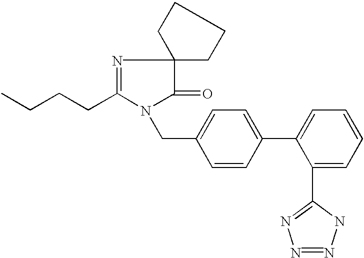

the compound with the designation SC-52458 of the following formula

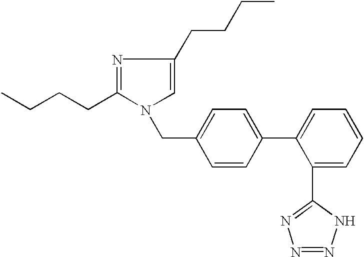

and the compound with the designation the compound ZD-8731 of the following formula

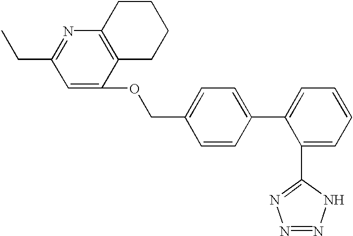

or, in each case, a pharmaceutically acceptable salt thereof.

Preferred ARBs are those agents which have been marketed, most preferred is valsartan or a pharmaceutically acceptable salt thereof.

Anti-hypertensive drugs within the scope of the present invention include, but are not limited to, calcium channel blockers (CCBs), angiotensin converting enzyme (ACE) inhibitors, diuretics, vasodilators, ARBs, α and β adrenergic blockers and combinations of the above, for example, ACE inhibitors plus one of CCBs and diuretics and α and β adrenergic blockers plus diuretics.

Examples of CCBs useful in the combinations of the present invention are selected from the group consisting of diltiazem, nifedipine, nitrendipine, nimodipine, niludipine, niguldipine, nicardipine, nisoldipine, amlodipine, felodipine, isradipine, ryosidine, verapamil, gallopamil and tiapamil or in each case a pharmaceutically acceptable salt thereof.

The class of ACE inhibitors comprises compounds having differing structural features. For example, mention may be made of the compounds which are selected from the group consisting alacepril, benazepril, benazeprilat, captopril, ceronapril, cilazapril, delapril, enalapril, enaprilat, fosinopril, imidapril, lisinopril, moveltopril, perindopril, quinapril, ramipril, spirapril, temocapril, and trandolapril, or, in each case, a pharmaceutically acceptable salt thereof.

Preferred ACE inhibitors are those agents which have been marketed, most preferred are benazepril and enalapril or pharmaceutically acceptable salt thereof.

The class of diuretics include carbonic anhydrase inhibitors such as diclorphenamide; loop diuretics such as bumetanide, torsemide, ethacrynic acid and furosemide; potassium-sparing diuretics such as spironolactone, triamterene and amiloride; and thiazides such as hydroflumethiazide, chlorothiazide, hydrochlorothiazide, methychlothiazide, metolazone and chlorthalidone or, in each case, a pharmaceutically acceptable salt thereof.

Vasodilators include nitroglycerin and isosorbide mono- and di-nitrate.

β adrenergic blocker include propranolol, bisoprolol and metoprolol.

Statins include atorvastatin, cerivastatin, fluvastatin, lovastatin, itavastatin, pravastatin, and simvastatin, or, in each case, a pharmaceutically acceptable salt thereof.

Preferred statins are those agents which have been marketed, most preferred are fluvastatin, simvastatin or a pharmaceutically acceptable salt thereof.

The term “pharmaceutically acceptable salts” or “ a pharmaceutically acceptable salt thereof” refer to salts prepared from pharmaceutically acceptable nontoxic acids or bases including inorganic acids and bases. Suitable pharmaceutically acceptable acid salts for the first agent and the co-agents of the present invention include acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like.

The pharmaceutical compositions of the present invention comprise the pharmaceutical combinations as described above plus a pharmaceutically acceptable carrier.

“SD associated with hypertension” as that term is used herein means the incidence of SD resulting from hypertension as well as from the medical treatment of hypertension with drugs irrespective of the presence of diabetes and hyperlipidemia.

“SD associated with hypertension and another condition, including but not limited to hyperlipidemia and diabetes” as that term is used herein means the incidence of SD resulting from these conditions.

The treatment of SD associated with hypertension and the treatment of SD associated with hypertension and another condition by methods described in the present invention may be demonstrated in the following pharmacological test:

An international, multicenter, double-blind, randomized, active-controlled trial, is conducted in approximately 14,000 patients with essential hypertension and moderate to high cardiovascular risk profiles. In this trial, valsartan or amlodipine are administered as monotherapy. Dosages, once a day are as follows: Valsartan is administered in 40, 80 or 160 mg; amlodipine is administered in 2.5, 5 or 10 mg.

For combination therapy, valsartan is administered in combination with one of amlodipine, simvastatin or hydrochlorothiazide (HCTZ). During the development of these combinations, valsartan is administered once (o.d.) or twice daily (b.i.d.) at 40, 80, 160 or 320 mg. Co-administered with valsartan is Amlodipine at a dose of 2.5, 5 or 10 mg; simvastatin at a dose of 20, 40 or 80 mg or HCTZ at a dose of 12.5 or 25 mg.

After the administration of the above monotherapies and combinations patients are evaluated for quality of life, including sexual function. Applicant has surprisingly found that the combinations described above achieve a therapeutic effect of lowering SD in the patients greater than the therapeutic effect achieved by the sum of the administration of the active ingredients separately.

Further, administration of pharmaceutical combinations of the invention have a therapeutic effect for: (i) reducing SD associated with hypertension and (ii) reducing SD associated with hypertension and another condition. The administration of these combinations also achieves a synergistic therapeutic effect for: (i) reducing SD associated with hypertension and (ii) reducing SD associated with hypertension and another condition which effect is greater than the sum of the therapeutic effect achieved by administration of the active ingredients separately.

To prepare the pharmaceutical compositions of the present invention, the active ingredients, or their pharmaceutically acceptable salts, racemates or enantiomers are combined in intimate admixture by mixing, blending or combining in any manner known to those of skill in the art, with a pharmaceutically acceptable carrier. The pharmaceutically acceptable carrier may take a wide variety of forms depending on the form of preparation desired for administration. As an example, the pharmaceutical compositions comprise of from about 0.1% to 90%, preferably of from about 1% to about 80% of the active ingredients.

Any suitable route of administration may be employed for providing a mammal with a therapeutically effective amount of the pharmaceutical combinations and compositions of the present invention. For example, oral, rectal, vaginal, topical, parental (subcutaneous, intramuscular, intravenous, transdermal) and like forms of administration may be employed. Dosage formulations include ointments, foams, gels, transdermal patches, tablets (both fractionable and non-fractionable), caplets, powders for inhalations, gel caps, capsules, elixirs, syrups, chewable tablets, lozenges, troches, dispersions, aerosols, solutions, fastdissolving wafers, suppositories or suspensions or other known and effective delivery methods.

Oral dosing is preferred. In preparing the compositions in oral dose form, any of the usual pharmaceutical carriers may be employed including any material, composition or vehicle, such as a liquid or solid filler, diluent, excipient, solvent or encapsulating material involved in carrying, formulating or transporting a chemical agent. Specific examples are water, glycols, oils, alcohols and the like in the case of oral liquid preparations. In oral solid forms solid carriers such as starches, sugars, kaolin, lubricants, binders, disintegrating agents and the like may be employed. Oral solid preparations are preferred over the oral liquid preparations. A preferred oral solid preparation is capsules and tablets, because of their ease of administration.

For parental compositions, the carrier will usually comprise sterile water, at least in large part, though other ingredients, to aid solubility for example, may be included. Injectable solutions, for example, may be prepared in which the carrier comprises PEG, saline solution, glucose solution or a mixture of saline and glucose solution. Injectable suspensions may also be prepared in which case appropriate liquid carriers, suspending agents and the like may be employed. In the compositions suitable for percutaneous administration, the carrier optionally comprises a penetration enhancing agent and/or a suitable wetting agent, optionally combined with suitable additives of any nature in minor proportions, which additives do not cause a significant deleterious effect on the skin. It is especially advantageous to formulate the aforementioned pharmaceutical compositions in dosage unit form for ease of administration and uniformity of dosage. Dosage unit form as used herein refers to physically discrete units suitable as unitary dosages, each unit containing a predetermined quantity of active ingredient(s) calculated to produce the desired therapeutic effect in association with the required pharmaceutical carrier.

The term “pharmaceutical combination” as used herein means a product that results from the mixing or combining of more than one active ingredient and the combination can be administered simultaneously or sequentially in any order, separately or in a fixed combination.

The dosage of the active compound can depend on a variety of factors, such as mode of administration, homeothermic species, age and/or individual condition.

Preferred dosages for the active ingredients of the pharmaceutical combination according to the present invention are therapeutically effective dosages, especially those which are commercially available.

The total daily dose range may be administered in a range of from about 0.01 mg to about 1000 mg. The daily dose range may be about 800, 600, 400, 200, 100, 50, 20, 10, 5, 1, .1 or 0 .01 mg. Preferably, a daily dose range should be between about 2.5 mg to about 540 mg, while most preferably, a daily dose range should be between about 5 mg to about 100 mg. It is preferred that the doses are administered o.d. or b.i.d. In managing the patient, the therapy should be initiated at a lower dose, perhaps about 5 mg to about 10 mg, and increased up to about 50 mg or higher depending on the patient's response. It may be necessary to use dosages outside these ranges in some cases as will be apparent to those skilled in the art. Further, it is noted that the clinician or treating physician will know how and when to interrupt, adjust, or terminate therapy in conjunction with individual patient response. The term “therapeutically effective amount” is encompassed by the abovedescribed molar ratio and dosage amounts and dose frequency schedule.

Normally, in the case of oral administration, an approximate daily dose of from about 1 mg to about 360 mg is to be estimated, e.g., for a patient of approximately 75 kg in weight.

The dosage of the active compound can depend on a variety of factors, such as mode of administration, homeothermic species, age and/or individual condition.

Valsartan, as a representative of the class of AT₁-receptor antagonists, is supplied in the form of suitable dosage unit form, for example, a capsule or tablet, and comprising a therapeutically effective amount, e.g., from about 20 mg to about 320 mg, of valsartan which may be administered to patients, preferably from about 80 mg to about 320 mg. The application of the active ingredient may occur up to three times a day, starting, e.g., with a daily dose of 20 mg or 40 mg of valsartan, increasing via 80 mg o.d. and further to 160 mg daily up to 320 mg o.d. Preferably, valsartan is applied b.i.d. with a dose of 80 mg or 160 mg, respectively, each. Corresponding doses may be taken, for example, in the morning, at mid-day or in the evening. Preferred is b.i.d. administration.

In case of calcium channel blockers, preferred dosage unit forms are, for example, tablets or capsules comprising, e.g., from about 2.5 mg to about 540 mg, preferably, when using amlodipine, for example, about 2.5 mg to about 10 mg administered o.d.; about 180 mg to about 540 mg of verapamil o.d.; about 120 mg to about 360 mg of diltiazem and about 2.5 mg to about 20 mg of isradipine o.d.

In case of ACE inhibitors, preferred dosage unit forms of ACE inhibitors are, for example, tablets or capsules comprising, e.g., from about 10 mg to about 80 mg, preferably 10, 20 or 40 mg, of benazepril and from about 2.5 mg to about 20 mg, preferably 2.5, 5, 10 or 20 mg, of enalapril.

In case of Beta blockers, preferred dosage unit forms are, for example, tablets or capsules comprising, e.g., from about 80 mg to about 640 mg of propranolol; from about 2.5 mg to about 20 mg of bisoprolol and from about 50 mg to about 400 mg of metoprolol.

In case of statins, preferred dosage unit forms are, for example, tablets or capsules comprising, e.g., from about 20 mg to about 80 mg of fluvastatin; from about 10 mg to about 80 mg of atorvastatin and from about 5 mg to about 80 mg of simvastatin, administered o.d.

Especially preferred are low dose combinations.

### EXAMPLES

The present invention is further described by the following examples. The examples are provided solely to illustrate the invention by reference to specific embodiments. These exemplification's, while illustrating certain specific aspects of the invention, do not portray the limitations or circumscribe the scope of the disclosed invention.

### Formulation Example 1

Film-coated tablets

| | Composition per | |
|---|---|---|
| Components | Unit (mg) | Standards |
| Granulation | | |
| Valsartan [= active ingredient] | 80.00 | |
| Microcrystalline cellulose/Avicel PH | 54.00 | NF, Ph. Eur |
| 102 | | |
| Crospovidone | 20.00 | NF, Ph. Eur |
| Colloidal anhydrous silica/Colloidal | 0.75 | Ph. Eur/NF |
| silicon dioxide/Aerosil 200 | | |
| Magnesium stearate | 2.5 | NF, Ph. Eur |
| Blending | | |
| Colloidal anhydrous silica/Colloidal | 0.75 | Ph. Eur/NF |
| silicon dioxide/Aerosil 200 | | |
| Magnesium stearate | 2.00 | NF, Ph. Eur |
| Coating | | |
| Purified water* | — | |
| DIOLACK pale red 00F34899 | 7.00 | |
| Total tablet mass | 167.00 | |

*Removed during processing.

The film-coated tablet is manufactured, e.g., as follows:

A mixture of valsartan, microcrystalline cellulose, crospovidone, part of the colloidal anhydrous silica/colloidal silicon dioxide/Aerosile 200, silicon dioxide and magnesium stearate is premixed in a diffusion mixer and then sieve through a screening mill. The resulting mixture is again pre-mixed in a diffusion mixer, compacted in a roller compacter and then sieve through a screening mill. To the resulting mixture, the rest of the colloidal anhydrous silica/colloidal silicon dioxide/Aerosile 200 are added and the final blend is made in a diffusion mixer. The whole mixture is compressed in a rotary tabletting machine and the tablets are coated with a film by using Diolack pale red in a perforated pan.

### Formulation Example 2

Film-coated tablets

| | Composition per | |
|---|---|---|
| Components | Unit (mg) | Standards |
| Granulation | | |
| Valsartan [= active ingredient] | 160.00 | |
| Microcrystalline cellulose/Avicel PH | 108.00 | NF, Ph. Eur |
| 102 | | |
| Crospovidone | 40.00 | NF, Ph. Eur |
| Colloidal anhydrous silica/colloidal | 1.50 | Ph. Eur/NF |
| silicon dioxide/Aerosil 200 | | |
| Magnesium stearate | 5.00 | NF, Ph. Eur |
| Blending | | |
| Colloidal anhydrous silica/colloidal | 1.50 | Ph. Eur/NF |
| silicon dioxide/Aerosil 200 | | |
| Magnesium stearate | 4.00 | NF, Ph. Eur |
| Coating | | |
| Opadry Light Brown 00F33172 | 10.00 | |
| Total tablet mass | 330.00 | |

The film-coated tablet is manufactured, e.g., as described in Formulation Example 1.

### Formulation Example 3

Film-coated tablets

| | Composition per | |
|---|---|---|
| Components | Unit (mg) | Standards |
| Core: Internal phase | | |
| Valsartan [= active ingredient] | 40.00 | |
| Silica, colloidal anhydrous (Colloidal | 1.00 | Ph. Eur, |
| silicon dioxide) [= Glidant] | | USP/NF |
| Magnesium stearate [= Lubricant] | 2.00 | USP/NF |
| Crospovidone (Disintegrant) | 20.00 | Ph. Eur |
| Microcrystalline cellulose [= Binding | 124.00 | USP/NF |
| agent] | | |
| External phase | | |
| Silica, colloidal anhydrous, (Colloidal | 1.00 | Ph. Eur, |
| silicon dioxide) [= Glidant] | | USP/NF |
| Magnesium stearate (Lubricant) | 2.00 | USP/NF |
| Film coating | | |
| Opadry ® brown OOF 16711* | 9.40 | |
| Purified Water** | — | |
| Total tablet mass | 199.44 | |

*The composition of the Opadry ® brown OOF16711 coloring agent is tabulated below.
**Removed during processing.

Opadry ® composition

| | Approximate |
|---|---|
| Ingredient | % Composition |
| Iron oxide, black (C.I. No. 77499, E 172) | 0.50 |
| Iron oxide, brown (C.I. No. 77499, E 172 | 0.50 |
| Iron oxide, red (C.I. No. 77491, E 172) | 0.50 |
| Iron oxide, yellow (C.I. No. 77492, E 172) | 0.50 |
| Macrogolum (Ph. Eur) | 4.00 |
| Titanium dioxide (C.I. No. 77891, E 171) | 14.00 |
| Hypromellose (Ph. Eur) | 80.00 |

The film-coated tablet is manufactured e.g. as described in Formulation Example 1.

### Formulation Example 4

Capsules

| | Composition |
|---|---|
| Components | per Unit (mg) |
| Capsules | |
| Valsartan [= active ingredient] | 80.00 |
| Microcrystalline cellulose | 25.10 |
| Crospovidone | 13.00 |
| Povidone | 12.50 |
| Magnesium stearate | 1.30 |
| Sodium lauryl sulphate | 0.60 |
| Shell | |
| Iron oxide, red (C.I. No. 77491, EC No. E 172) | 0.123 |
| Iron oxide, yellow (C.I. No. 77492, EC No. E 172) | 0.123 |
| Iron oxide, black (C.I. No. 77499, EC No. E 172) | 0.245 |
| Titanium dioxide | 1.540 |
| Gelatin | 74.969 |
| Total tablet mass | 209.50 |

The tablet is manufactured, e.g., as follows:

### Granulation/Drying

Valsartan and microcrystalline cellulose are spray-granulated in a fluidized bed granulator with a granulating solution consisting of povidone and sodium lauryl sulphate dissolved in purified water. The granulate obtained is dried in a fluidized bed dryer.

### Milling/Blending

The dried granulate is milled together with crospovidone and magnesium stearate. The mass is then blended in a conical screw type mixer for approximately 10 minutes.

### Encapsulation

The empty hard gelatin capsules are filled with the blended bulk granules under controlled temperature and humidity conditions. The filed capsules are dedusted, visually inspected, weight checked and quarantined until by Quality assurance department.

### Formulation Example 5

Capsules

| | Composition |
|---|---|
| Components | per Unit (mg) |
| Capsules | |
| Valsartan [= active ingredient] | 160.00 |
| Microcrystalline cellulose | 50.20 |
| Crospovidone | 26.00 |
| Povidone | 25.00 |
| Magnesium stearate | 2.60 |
| Sodium lauryl sulphate | 1.20 |
| Shell | |
| Iron oxide, red (C.I. No. 77491, EC No. E 172) | 0.123 |
| Iron oxide, yellow (C.I. No. 77492, EC No. E 172) | 0.123 |
| Iron oxide, black (C.I. No. 77499, EC No. E 172) | 0.245 |
| Titanium dioxide | 1.540 |
| Gelatin | 74.969 |
| Total tablet mass | 342.00 |

The formulation is manufactured, e.g., as described in Formulation Example 4.

### Formulation Example 6

Hard gelatin capsule

| | Composition |
|---|---|
| Components | per Unit (mg) |
| Capsules | |
| Valsartan [= active ingredient] | 80.00 |
| Sodium laurylsulphate | 0.60 |
| Magnesium stearate | 1.30 |
| Povidone | 12.50 |
| Crospovidone | 13.00 |
| Microcrystalline cellulose | 21.10 |
| Total tablet mass | 130.00 |

### Examples 7 to 11

| | Example | | | | |
|---|---|---|---|---|---|
| | 7 | 8 | 9 | 10 | 11 |
| | Amount | Amount | Amount | Amount | Amount |
| | per Unit | per Unit | per Unit | per Unit | per Unit |
| Components | (mg) | (mg) | (mg) | (mg) | (mg) |

Granulation

| | | | | | |
|---|---|---|---|---|---|
| Valsartan Drug | 80.000 | 160.000 | 40.000 | 320.000 | 320.000 |
| Substance | | | | | |
| Microcrystalline | 54.000 | 108.000 | 27.000 | 216.000 | 216.000 |
| Cellulose (NF, | | | | | |
| Ph. Eur.)/Avicel | | | | | |
| PH 102 | | | | | |
| Crospovidone | 15.000 | 30.000 | 7.500 | 80.000 | 60.000 |
| (NF, Ph. Eur.) | | | | | |
| Colloidal | 1.500 | 3.000 | 0.750 | 3.000 | 6.000 |
| Anhydrous | | | | | |
| Silica (Ph. | | | | | |
| Eur.)/Colloidal | | | | | |
| Silicon Dioxide | | | | | |
| (NF)/Aerosil 200 | | | | | |
| Magnesium | 3.000 | 6.000 | 1.500 | 10.000 | 12.000 |
| Stearate | | | | | |
| (NF, Ph. Eur.) | | | | | |
| Blending | | | | | |
| Colloidal | — | — | — | 3.000 | — |
| Anhydrous | | | | | |
| Silica (Ph. | | | | | |
| Eur.)/Colloidal | | | | | |
| Silicon Dioxide | | | | | |
| (NF)/Aerosil 200 | | | | | |
| Magnesium | 1.500 | 3.000 | 0.750 | 8.000 | 6.000 |
| Stearate, NF, Ph. | | | | | |
| Eur. | | | | | |
| Core Weight/mg | 155.000 | 310.000 | 77.500 | 640.000 | 620.000 |
| Coating | — | — | 3.800 | 15.000 | 16.000 |

### Example 12

Hard gelatin capsule

| Components | Amount per Unit (mg) |
|---|---|
| Capsule | |
| Fluvastatin Sodium* | 21.481** |
| Calcium Carbonate | 62.840 |
| Sodium Bicarbonate | 2.000 |
| Microcrystalline Cellulose | 57.220 |
| Pregelatinized Starch | 41.900 |
| Purified Water*** | Q.S. |
| Magnesium Stearate | 1.050 |
| Talc | 9.430 |
| Target Capsule Fill Weight | 195.92 |
| Capsule Shell | |
| Hard gelatin Capsule Shell | 48.500 |
| Branding Ink (pre-printed) | |
| White Ink | Trace |
| Red Ink | Trace |
| Target Capsule Weight | 244.42 |

*Includes a 2% overage for moisture.
***20 mg of free acid is equivalent to 21.06 mg Na salt.
***Partially removed during processing.

### Example 13

Hard gelatin capsule

| Components | Amount per Unit (mg) |
|---|---|
| Capsule | |
| Fluvastatin Sodium | 42.962*** |
| Calcium Carbonate | 125.680 |
| Sodium Bicarbonate | 4.000 |
| Microcrystalline Cellulose | 114.440 |
| Pregelatinized Starch | 83.800 |
| Purified Water*** | Q.S. |
| Magnesium Stearate | 2.100 |
| Talc | 18.860 |
| Target Capsule Fill Weight | 391.840 |
| Capsule Shell | |
| Hard gelatin Capsule Shell | 76.500 |
| Branding ink (pre-printed) | |
| White Ink | Trace |
| Red Ink | Trace |
| Target Capsule Weight | 468.34 |

*Includes a 2% overage for moisture.
**20 mg of free acid equivalent to 21.06 mg Na salt.
***Partially removed during processing.

### Example 14

Round, slightly bi-convex, film-coated tablets with beveled edges

| Components | Amount per Unit (mg) |
|---|---|
| Table Core | |
| Fluvastatin Sodium* | 84.24** |
| Cellulose Microcrystalline/Microcrystalline | 111.27 |
| cellulose fine powder | |
| Hypromellose/Hydroxypropyl methyl cellulose | 97.50 |
| (Methocel K100LVP CR; HPMC100 cps) | |
| Hydroxypropyl cellulose (Klucel HXF) | 16.25 |
| Potassium hydrogen carbonate/Potassium | 8.42 |
| bicarbonate | |
| Povidone | 4.88 |
| Magnesium stearate | 2.44 |
| Core Tablet Weight | 325.00 |
| Coating | |
| Coating premix-Opadry Yellow (00F22737) | 9.75 |
| Total Weight | 334.75 |
| Water, purified*** | Q.S. |

*84.24 mg of the sodium salt of fluvastatin is equivalent to 80 mg of fluvastatin free acid.
**To be adjusted for moisture (LOD).
***Removed during processing.

### Example 15

Round, biconvex, beveled-edged, film-coated tablets

| | Unit | Unit | Unit | Unit |
|---|---|---|---|---|
| | wt./Vol. | wt./Vol. | wt./Vol. | wt./Vol. |
| Components | (mg) | (mg) | (mg) | (mg) |
| Benazepril Hydrochloride | 5.00 | 10.00 | 20.00 | 40.00 |
| Lactose Monohydrate, NF | 142.00 | 132.00 | 117.00 | 97.00 |
| Pregelatinized Starch, NF | 8.00 | 8.00 | 8.00 | 8.00 |
| Colloidial Silicon Dioxide, NF | 1.00 | 1.00 | 1.00 | 1.00 |
| (Cab-O-Sil, M-5) | | | | |
| Crospovidone, NF | 3.00 | 3.00 | 3.00 | 3.00 |
| Microcrystalline Cellulose, NF | 18.00 | 18.00 | 18.00 | 24.25 |
| Hydrogenated Castor Oil, NF | 8.00 | 8.00 | | |
| Magnesium Stearate, NF | | | 8.00 | 1.75 |
| Color: | | | | |
| Yellow-Brown (suspension) | — | | | 0.50 |
| Red-Brown (suspension) | | 2.00 | 0.50 | |
| Purified Water, USP | Trace | trace | trace | trace |
| Opadry Color: | | | | |
| Yellow | 8.38 | 8.38 | | |
| Pink | | | 8.38 | 8.38 |
| Total | 193.38 | 190.38 | 183.88 | 183.88 |

Although the present invention has been described in considerable detail with reference to certain preferred versions thereof, other versions are possible without departing from the spirit and scope of the preferred versions contained herein. All references and Patents (U.S. and others) referred to herein are hereby incorporated by reference in their entirety as if set forth herein in full.

What is claimed is:

1. A method of achieving a therapeutic effect for treating a patient suffering from sexual dysfunction (SD) associated with hypertension comprising administering a therapeutically effective amount of a pharmaceutical combination comprising as active ingredients:
(i) an ARB or a pharmaceutically acceptable salt thereof and
(ii) (a) an anti-hypertensive drug or a pharmaceutically acceptable salt thereof or (b) a statin or a pharmaceutically acceptable salt thereof to the patient.

2. A method of achieving a therapeutic effect for treating a patient suffering from SD associated with hypertension and another condition comprising administering a therapeutically effective amount of a pharmaceutical combination comprising as active ingredients:
(i) an ARB or a pharmaceutically acceptable salt thereof and
(ii) (a) an anti-hypertensive drug or a pharmaceutically acceptable salt thereof or (b) a statin or a pharmaceutically acceptable salt thereof to the patient.

3. The method of claim 1 wherein the ARB, anti-hypertensive drug and HG-CoA reductase inhibitor include pharmaceutically acceptable racemates or enantiomers thereof.

4. The method of claim 1 wherein the ARB is selected from the group consisting of valsartan, losartan, candesartan, eprosartan, irbesartan, saprisartan, tasosartan, telmisartan, E-1477, SC-52458 and ZD-8731.

5. The method of claim 1 wherein the ARB is valsartan.

6. The method of claim 5 wherein the anti-hypertensive drug is selected from the group consisting of one or more of CCBs, ACE inhibitors, diuretics, vasodilators, ARBs, α and β adrenergic blockers, ACE inhibitors in combination with CCBs, diuretics, α and β adrenergic blockers, and diuretics.

7. The method of claim 6 wherein the CCBs are selected from the group consisting of diltiazem, nifedipine, nitrendipine, nimodipine, niludipine, niguldipine, nicardipine, nisoldipine, amlodipine, felodipine, isradipine, ryosidine, verapamil, gallopamil and tiapamil.

8. The method of claim 6 wherein the ACE inhibitors are selected from the group consisting of alacepril, benazepril, benazeprilat, captopril, ceronapril, cilazapril, delapril, enalapril, enaprilat, fosinopril, imidapril, lisinopril, moveltopril, perindopril, quinapril, ramipril, spirapril, temocapril, and trandolapril.

9. The method of claim 6 wherein the diuretics are selected from the group consisting of carbonic anhydrase inhibitors, combination diuretics, loop diuretics, potassium-sparing diuretics and thiazides.

10. The method of claim 9 wherein the carbonic anhydrase is diclorphenamide.

11. The pharmaceutical combination of claim 9 wherein the loop diuretics are selected from the group consisting of bumetanide, torsemide, ethacrynic acid and furosemide.

12. The pharmaceutical combination of claim 9 wherein the potassium-sparing diuretics are selected from the group consisting of spironolactone, triamterene and amiloride.

13. The pharmaceutical combination of claim 9 wherein the thiazides are selected from the group consisting of hydroflumethiazide, chlorothiazide, hydrochlorothiazide, methychlothiazide, metolazone and chlorthalidone.

14. The pharmaceutical combination of claim 6 wherein the vasodilators are selected from the group consisting of nitroglycerin and isosorbide mono-and di-nitrate.

15. The pharmaceutical combination of claim 6 wherein the β adrenergic blockers are selected from the group consisting of propranolol, bisoprolol and metoprolol.

16. The method of claim 1 wherein the pharmaceutical combination further comprises pharmaceutically acceptable carriers.
